(19) United States
(12) Patent Application Publication
(10) Pub. No.: US 2022/0093220 A1
(43) Pub. Date: Mar. 24, 2022

(54) SYSTEM AND METHOD FOR PATIENT ASSESSMENT USING DISPARATE DATA SOURCES AND DATA-INFORMED CLINICIAN GUIDANCE VIA A SHARED PATIENT/CLINICIAN USER INTERFACE

(71) Applicant: Seth Feuerstein, New Haven, CT (US)

(72) Inventor: Seth Feuerstein, New Haven, CT (US)

(21) Appl. No.: 17/477,671

(22) Filed: Sep. 17, 2021

Related U.S. Application Data
(60) Provisional application No. 63/080,389, filed on Sep. 18, 2020.
(60) Provisional application No. 63/210,796, filed on Jun. 15, 2021.

(51) Int. Cl. G16H 10/20; G16H 80/00; G16H 10/60; G10L 25/66; G06K 9/00

(52) U.S. Cl. CPC G16H 10/20; G16H 80/00; G06K 9/00268; G10L 25/66; G16H 10/60

(57) ABSTRACT

A system and method for patient assessment using disparate data sources and data-informed clinician guidance via a shared patient/clinician user interface. The interface can be offered in person, with both patient and clinician in the same physical environment, or with each of them in different locations, using computerized devices linked via a communications network and/or a telehealth interface. The system guides the clinician in a collaborative way that ensures fidelity to a proper and high-quality clinical outcome, while retaining clinician-patient interactions and engagement. Accordingly, not every clinician is required to be a sub-specialist in a particular condition, such as suicide care, and a non-specialist clinician can perform an effective patient assessment because of the guidance provided by the system.

### CROSS-REFERENCE TO RELATED APPLICATIONS

This application claims the benefit of priority, under 35 U.S.C. 119(e), of U.S. Provisional Patent Application No. 63/080,389, filed Sep. 18, 2020, and U.S. Provisional Patent Application No. 63/210,796, filed Jun. 15, 2021, the entire disclosures of both of which are hereby incorporated herein by reference.

### FIELD OF THE INVENTION

The present invention relates generally to patient assessment and intervention for medical diagnostic, tracking and treatment purposes, and more specifically, to a computerized system and method for these using disparate data sources and data-informed clinician guidance via a shared patient/clinician user interface provided by the system.

### DISCUSSION OF RELATED ART

Clinical patient interactions are performed in a variety of settings in an attempt to measure a person's behavioral status and functional situations across a broad range of clinical domains such as mood, anxiety, psychosis, suicidality, obsessions, compulsions, addictions and medication response for these as well. By way of example, a person arriving at an Emergency Room (ER) of a hospital may be submitted to a clinical patient assessment to screen the patient for suicidality.

Such clinical patient assessments are intended to be administered by trained clinicians, requiring face to face human interactions and limiting how often these assessments can be performed. Even in the most intensive settings, such as an inpatient unit, suicidality evaluations such as these occur infrequently and rarely with a high level of fidelity to what has been proven to work. Generally, these assessments involve a dialogue between the clinician and patient, with the clinician posing questions, the patient offering responses, and the clinician using experience and judgment to guide the clinician's line of inquiry. The patient may provide accurate, known false, unknown false and/or inconsistent responses. Accordingly, these evaluations are somewhat subjective and require substantial experience and training to perform them most effectively. Accordingly, the results of suicidality evaluations can vary greatly due to improper or inadequate training, lack of experience in performing these evaluations and/or other subjective factors, and thus the results may vary for a single patent as a function of who performs the evaluation. Clinical patient assessments screening for other medical issues face similar problems to a greater or lesser degree. This is problematic, as it tends to lead to inadequate frequency and effectiveness of patient screening, as there is often a shortage of time for performing such tasks and/or a shortage of properly trained personnel for performing these tasks.

Further, in the event that a patient screens positively for suicidality, this triggers the need for certain documentation of the assessment, the conclusions, a safety plan, etc. in accordance with hospital procedures, best practices and/or governing and/or thought-leading bodies, such as the Joint Commission for Hospitals. As a practical matter, when clinicians are left to perform open ended, free-form documentation, there are ample opportunities for improper or incomplete processes and/or documentation as there is very little procedurally that effectively ensures that such documentation is completed, and completed accurately/adequately.

Where new attempts have been made to streamline clinical patient assessments and ensure fidelity to what has been proven to work by automating patient interviews, such attempts have generally involved simple and straightforward fact-gathering via a pre-defined questionnaire displayed via a tablet PC or other computing device, as somewhat of an electronic/software-based replacement for completion of a paper/written questionnaire—much like gathering a simple medical history requiring entry of name, age, sex and other demographic information and providing simple (e.g., Yes/No) responses to simple questions (e.g., Have you ever been diagnosed with [condition]?). This is inadequate for proper clinical patient assessments, particularly when one assesses and then needs to gather a nuanced patient narrative to screen for suicidality or other conditions in which the line of questioning tends to be less well-defined, and more reactive to patient responses.

What is needed is a solution for performing clinical patient assessments that is more robust and flexible than a pre-defined questionnaire, that streamlines the patient assessment process while also retaining the option for human clinician judgment and involvement, and while reducing the impact of false, misleading and/or inconsistent responses from patients being assessed, such that a sub-specialist for a particular condition is not required in every instance to perform an effective patient assessment. Also needed is a system that can gather data about each interaction and link this data to longer term outcomes in data sets from health systems and payers to apply improvements regularly to previously static approaches.

### SUMMARY

The present invention provides a system and method for patient assessment using disparate data sources and data-informed clinician guidance via a shared patient/clinician user interface. In this manner, not every clinician is required to be a sub-specialist in a particular condition, such as suicide care, and a non-specialist clinician can perform an effective patient assessment because the system guides the clinician in a collaborative way that ensures fidelity to a proper and high-quality clinical outcome, while retaining clinician-patient interactions and engagement. The interface can be offered in person with both patient and clinician in the same physical environment or with each of them in different locations, using computerized devices linked via a communications network and/or a telehealth interface.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a schematic diagram of an exemplary special-purpose Patient Assessment and Clinician Guidance System computing device in accordance with an exemplary embodiment of the present invention;

FIG. 3 illustrates an exemplary graphical user interface displayable by the Patient Assessment and Clinician Guidance System for providing a shared patient/clinician session via a single display screen of a single computing device in accordance with an exemplary embodiment of the present invention;

FIG. 4 illustrates an exemplary graphical user interface displayable by the Patient Assessment and Clinician Guidance System for providing a shared patient/clinician session via a multiple display screens of multiple computing devices in accordance with an alternative exemplary embodiment of the present invention; and

FIGS. 5-20 illustrate another exemplary graphical user interface displayable by the Patient Assessment and Clinician Guidance System for providing a shared patient/clinician session via a multiple display screens of multiple computing devices in accordance with an alternative exemplary embodiment of the present invention.

### DETAILED DESCRIPTION

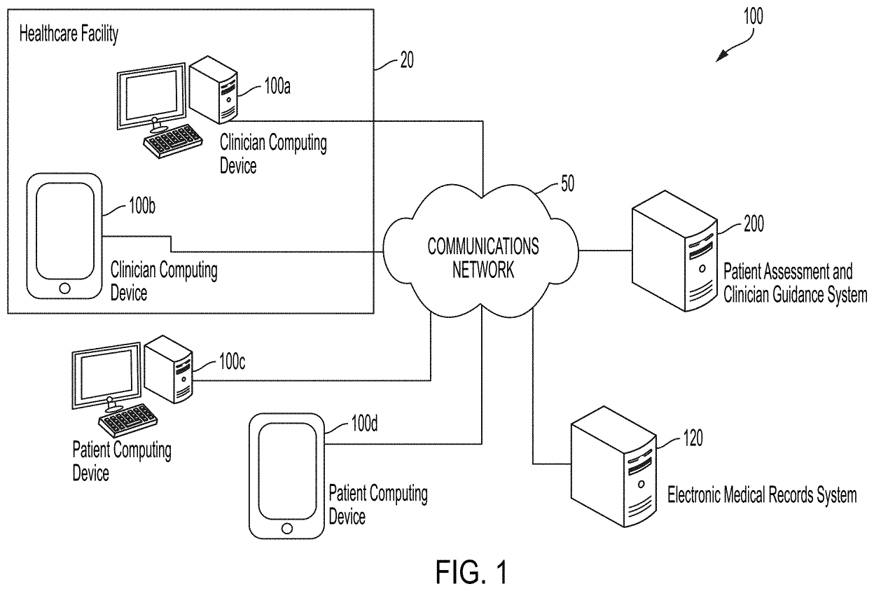

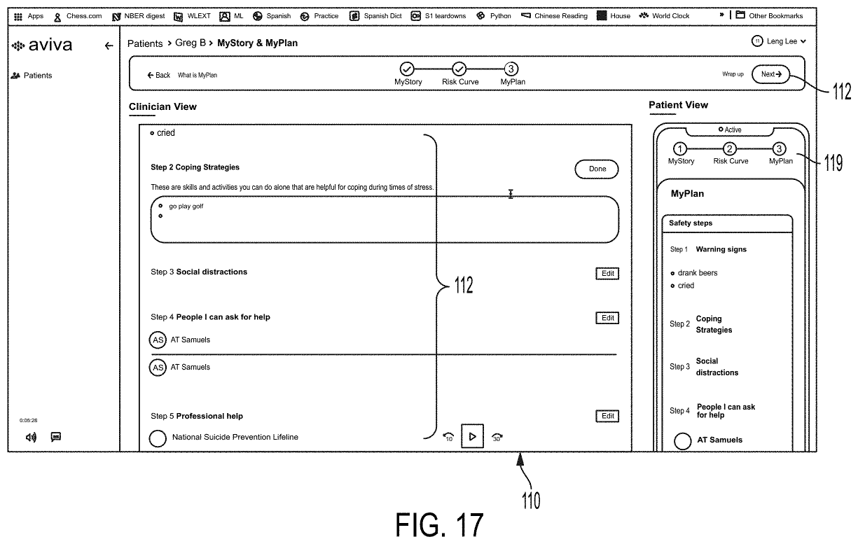

According to illustrative embodiment(s) of the present invention, various views are illustrated in FIG. 1-20 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

The present invention provides a system and method configured to perform clinical patient assessments that are more robust and flexible than a pre-defined questionnaire, and that are streamlined and semi-automated. Further, the system and method may capture and interpret passively-provided input to reduce the impact of false, misleading and/or inconsistent responses from patients being assessed. Further still, the system and method may use input provided actively via patient responses, and passively-provided input, such as computerized analyses of a patient's facial features/expressions and/or voice/vocalizations, as well as data gleaned and/or interpreted from patient medical records, to inform and guide a clinician, and facilitate and enhance clinician assessment, to retain a component of human clinician judgment and involvement, and to promote compliance with predetermined/best practices for questioning patients, guiding discussion, etc. Still further, the system at least partially-automates the documentation process by recording patient responses and passively-provided input and expressing it as output, as well as guiding the clinician through a supplemental documentation process. Further, the system may provide a shared interface allowing the clinician and patient to have a high-degree of collaboration in capturing and documenting information relevant to a patient assessment by providing for entry of data by a clinician and real-time/contemporaneous review of such data entry by the patient by providing a shared interface in which both the clinician and the patient can view documentation created by the clinician.

### System Environment

An exemplary embodiment of the present invention is discussed below for illustrative purposes. FIG. 1 is a system diagram showing an exemplary network computing environment 10 in which the present invention may be employed. As shown in FIG. 1, the exemplary network environment 10 includes conventional computing hardware and software for communicating via a communications network 50, such as the Internet, etc., using Caregiver Computing Devices 100a, 100b and/or Patient Computing Devices 100c, 100d, which may be, for example, one or more personal computers/PCs, laptop computers, tablet computers, smartphones, or other computing devices.

In accordance with a certain aspect of the present invention, the Clinician Computing Device (that may be used by the patient) and/or the Patient Computing Device (that may be used by the patient) includes a camera, such as a user-facing camera of a type often found in conventional smartphones, tablet PCs, laptops, etc. For example, the camera may be used to capture image data observed from the patient's face during use of the computing device. Any suitable conventional camera may be used for this purpose.

In accordance with another aspect of the present invention, the Clinician Computing Device (that may be used by the patient) and/or the Patient computing Device (that may be used by the patient) includes a microphone, such as a microphone of a type often found in conventional smartphones, tablet PCs, laptops, etc. For example, the microphone may be used to capture speech or other sound data observed from the patient's vocalizations during use of the computing device. Any suitable conventional microphone may be used for this purpose.

The network computing environment 10 may also include conventional computing hardware and software as part of a conventional Electronic Health Records System and/or an Electronic Medical Records System, such as an EPIC or Cerner or ALLSCRIPTS system, which are referred to collectively herein as an Electronic Medical Records (EMR) System 120. The EMR System 120 may interface with the Caregiver and/or Patient Computing Devices 100a, 100b, 100c, 100d and/or other devices as known in the art. These systems may be existing or otherwise generally conventional systems including conventional software and web server or other hardware and software for communicating via the communications network 50. Consistent with the present invention, these systems may be configured, in conventional fashion, to communicate/transfer data via the communications network 50 with the Patient Assessment and Clinician Guidance (PACG) System 200 in accordance with and for the purposes of the present invention, as discussed in greater detail below.

In accordance with the present invention, the network computing environment 100 further includes the Patient Assessment and Clinician Guidance (PACG) System 200. In this exemplary embodiment, the PACG System 200 is operatively connected to the Caregiver Computing Devices 100a, 100b and/or Patient Computing Devices 100c, 100d, and to the EMR System 120, for data communication via the communications network 50. For example, the PACG 200 may gather patient-related data from the Caregiver and/or Patient Computing Devices 100a, 100b, 100c, 100d via the communications network 50. Further, for example, the PACG 200 may gather via the communications network 50 medical/health records data from the EMR System 120 via the communications network 50. The gathered data may be used to perform analyses of the patient's current activities and/or the patient's past health/medical records, and the results of such analyses may be used by the PACG 200 to cause display of corresponding information via one or more graphical user interfaces at the Caregiver and/or Patient Computing Devices 100a, 100b, 100c, 100d by communication via the communications network 50. Hardware and software for enabling communication of data by such devices via such communications networks are well known in the art and beyond the scope of the present invention, and thus are not discussed in detail herein.

Accordingly, for example, a clinician may be assisted in conducting a clinical patient assessment by a patient's use of a clinician's Clinician Computing device 100a, 100b, e.g., within a hospital or other healthcare facility 20. Alternatively, for example, a clinician may be assisted in conducting a clinical patient assessment by a patient's use of a patient's Patient Computing Device 100c, 100d (either inside or outside a hospital or other healthcare facility 20), while the clinician uses the clinician's Clinician Computing device 100a, 100b, e.g., either inside or outside a hospital or other healthcare facility 20. In any case, the device 100a, 100b, 100c, 100d displays textual questions and/or other prompts to the patient, and the patient may interact with the device 100a, 100b, 100c, 100d to provide to the device, in an active fashion, input responsive to the questions/prompts—e.g., by touching a touchscreen, using a stylus, typing on a keyboard, manipulating a mouse, etc. The questions/prompts may be presented based on questions stored in the memory of the device and/or in the PACG 200. Preferably, those questions/prompts are defined in predetermined fashion, based on industry guidelines, thought leader guidance, experienced clinicians, or the like, so that they are consistent with best practices for gathering information from the patient. In certain embodiments, the sequence is static, such that the questions/prompts are presented in a predefined sequence that is consistent across patients and sessions. In a preferred embodiment, the sequence is dynamic, such that questions are presented according to predefined logic, but in a fluid sequence that may vary from person to person or session to session, based on input provided actively by the patient, and/or based on input gathered passively from the patient, e.g., using branched logic, machine learning, artificial intelligence, or other approaches to select next questions/prompts based at least in part on information provided by or gathered from the patient. The selection and/or development of next questions/prompts to be displayed by the user may be performed by the PACG 200. This may be done in various ways. For example, the PACG 200 may retrieve health/medical record data for the patient from the EMR System 120, and use branched logic, machine learning, artificial intelligence, or other approaches to select next questions/prompts based at least in part on information gathered from the EMR System 120.

By way of alternative example, the PACG 200 may obtain facial image data captured by a camera of the computing device used by the patient during the clinical assessment session, and the PACG 200 may process and interpret that data, and use branched logic, machine learning, artificial intelligence, or other approaches to select next questions/prompts based at least in part on an interpretation of the facial image data captured by the camera.

By way of yet another alternative example, the PACG 200 may obtain vocalization/voice data captured by a microphone of the computing device used by the patient during the clinical assessment session, and the PACG 200 may process and interpret that data, and use branched logic, machine learning, artificial intelligence, or other approaches to select next questions/prompts based at least in part on an interpretation of the vocalization/voice data captured by the camera.

Additionally, data captured from active/explicit input from the patient in response to questions/prompts displayed at the computing device, and data captured from passive input (such as data from the EMR System 120, or from interpretation of facial image or vocalization/voice data) may be further used for another purpose. Specifically, such data may be used to display discussion questions, discussion topics, health/medical history facts, or other prompts to the clinician via either the Clinician Computing Device 100a/100b or the Patient Computing Device 100c/100d. These prompts to the clinician provide additional information to the clinician that the clinician may use during the patient clinical assessment session to interact with the patient to perform a more accurate patient clinical assessment. By way of example, these prompts may be displayed in a subtle and/or coded fashion. For example, this may be appropriate when the patient and clinician are conducting a shared session and sharing a single device having a single display screen, such that all prompts to the clinician will be readily visible to the patient. By way of alternative example, these prompts may be displayed in an explicit fashion. For example, this may be appropriate when the patient and clinician are conducting a shared session without sharing a single device, such that each of the patient and clinician are using separate devices having separate display screens, such that all prompts to the clinician (on the computing device used by the clinician) will not be readable visible to the patient (on the computing device used by the patient). Accordingly, interview responses provided directly from the patient are supplemented with passively-gathered patient data, and used to guide the questioning of the patient via the computing device and/or to guide the clinician in interacting with the patient, to perform better patient clinical assessments.

Additionally, data may be captured from active dialog between the clinician and patient and/or explicit input from the patient (e.g., in response to questions/prompts displayed at either computing device and/or verbal questions presented to the patient by the clinician), and data may be, by the patient and the Patient Computing device and/or by the Clinician at the Clinician Computing Device), and the system may provide a shared user interface allow the patient and the clinician, at their respective devices, to view and review information input by the Clinician and displayed at both devices contemporaneously, to allow for a highly-collaborate session between the clinician and the patient, in real-time, via multiple user interfaces of multiple computing devices.

The data captured from the system is preferably persisted in the system's storage (e.g., at the PACG 200 or at local hardware, e.g., at the hospital 20) and then further transmitted to a cloud computing system (e.g., PACG 200) so that data may be later used to create reports or otherwise document the patient clinical assessment.

### Patient Assessment and Clinician Guidance System

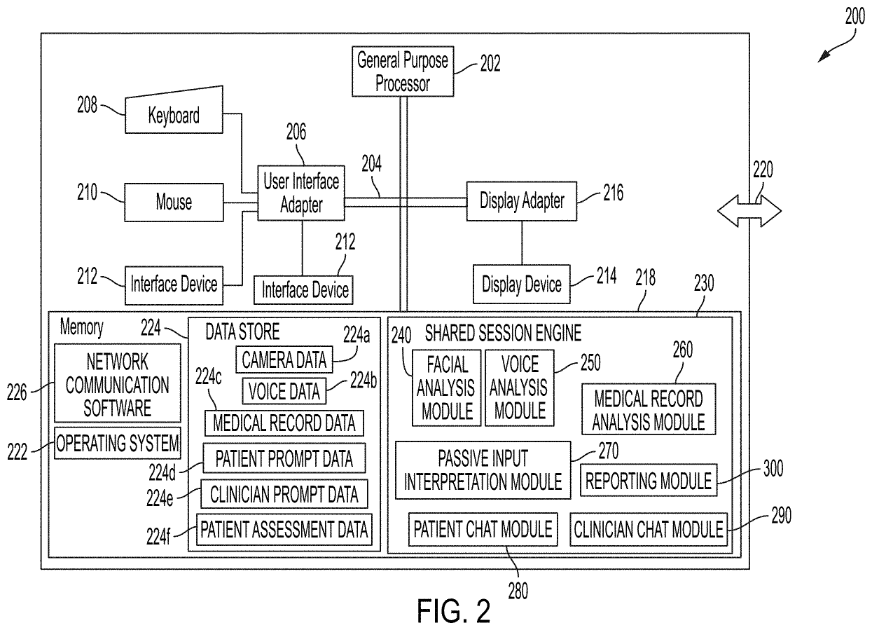

FIG. 2 is a block diagram showing an exemplary Patient Assessment and Clinician Guidance (PACG) System 200 in accordance with an exemplary embodiment of the present invention. The PACG System 200 is a special-purpose computer system that includes conventional computing hardware storing and executing both conventional software enabling operation of a general-purpose computing system, such as operating system software 222, network communications software 226, and specially-configured computer software for configuring the general purpose hardware as a special-purpose computer system for carrying out at least one method in accordance with the present invention. By way of example, the communications software 226 may include conventional web server software, and the operating system software 22 may include iOS, Android, Windows, Linux software.

Accordingly, the exemplary PACG System 200 of FIG. 2 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a camera, microphone, touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The PACG System 200 may communicate with other computers or networks of computers, for example via a communications channel, network card or modem 220. The PACG system 200 may be associated with such other computers in a local area network (LAN) or a wide area network (WAN), and may operate as a server in a client/server arrangement with another computer, etc. Such configurations, as well as the appropriate communications hardware and software, are known in the art.

The PACG System 200 is specially-configured in accordance with the present invention. Accordingly, as shown in FIG. 2, the PACG System 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the PACG System 200 includes, in accordance with the present invention, a Shared Session Engine (SSE) 230, shown schematically as stored in the memory 218, which includes a number of additional modules providing functionality in accordance with the present invention, as discussed in greater detail below. These modules may be implemented primarily by specially-configured software including microprocessor—executable instructions stored in the memory 218 of the PACG System 200. Optionally, other software may be stored in the memory 218 and and/or other data may be stored in the data store 224 or memory 218.

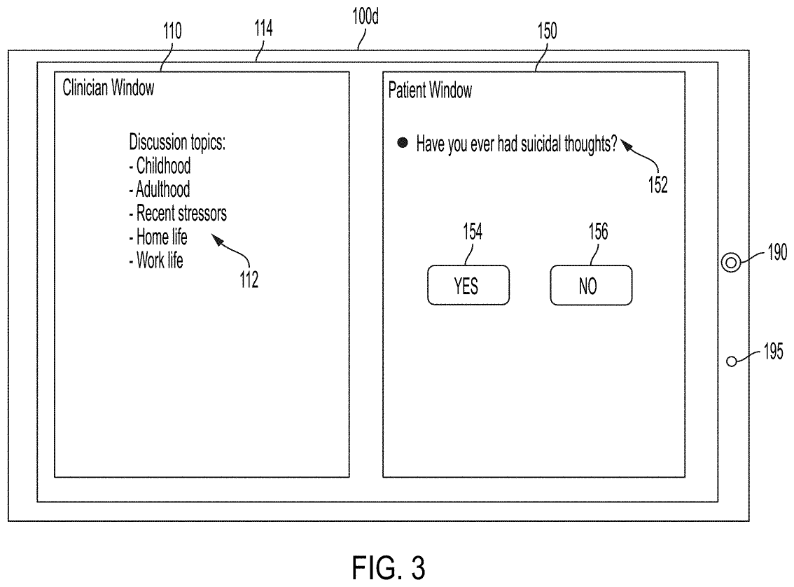

The exemplary embodiment of the PACG System 200 shown in FIG. 2 includes camera data 224a stored in the data store 224 of the PACG 200. The camera data 224a may be image data captured by a camera-type interface device 190 of a patient or caregiver computing device 100a, 100b, 100c, 100d, and in particular image data depicting the face of a user during the user's operation of the computing device 100a, 100b, 100c, 100d during a clinical patient assessment session. Accordingly, image data depicting the patient's face may be captured during the patient's operation of the computing device 100a, 100b, 100c, 100d to answer or respond to a prompt 154 displayed to the patient in a graphical user interface window 150 on a display device 114 of the computing device, e.g., 100d, as will be appreciated from FIG. 3. In this embodiment, the SSE 230 further includes a Facial Analysis Module 240. The Facial Analysis Module 240 is responsible for processing the camera data, e.g., to identify and/or analyze image features, facial expressions, facial muscle movements and/the like that are useful for drawing conclusions about the patient's then-current behavior, according to predetermined logic. By way of example, the camera data may be processed to identify and/or analyze image features, etc. useful for drawing conclusions about the patient's truthfulness, distress level, etc. For example, although there are many more options, if facial expression/image data indicates uncertainty, the system can alert the clinician to inquire further or to ask about how certain the patient is. If the facial expression/image data indicates that the patient is feeling overwhelmed, the clinician can be alerted with this information and also be provided with suggestions about what to say or ask. If the facial expression/image data indicates irritability, the system can let the clinician know and offer the clinician options of text to be spoken by the clinician to guide the patient/clinician interaction session, such as to ask whether the patient might need a break, whether the patient feels OK or if something is troubling the patient, etc. By way of further example, if the system determines that the patient is not being truthful when indicating that the patient does not intend to harm himself/herself, the system may provide an alert to the clinician so that the clinician can take appropriate action. The system thereby enhances and augments the clinician and patient interaction session and increases the clinician's human perceptions of affect and mood in the interaction.

The exemplary embodiment of the PACG System 200 shown in FIG. 2 also includes voice data 224b stored in the data store 224 of the PACG 200. The voice data 224b may be voice/vocalization data captured by a microphone-type interface device 195 of a patient or caregiver computing device 100a, 100b, 100c, 100d, and in particular voice/vocalizations of the user during the user's operation of the computing device 100a, 100b, 100c, 100d during a clinical patient assessment session. Accordingly, voice data depicting the patient's face may be captured during the patient's operation of the computing device 100a, 100b, 100c, 100d to answer or respond to a prompt 154 displayed to the patient in a graphical user interface window 150 on a display device 114 of the computing device, e.g., 100d, as will be appreciated from FIG. 3. In this embodiment, the SSE 230 further includes a Voice Analysis Module 250. The Voice Analysis Module 250 is responsible for processing the voice data, e.g., to identify and/or analyze, words and language used, presence or absence of voice, tone of voice, word choice, length of words chosen, speed of speech, quantity of words, length of sentences, use of neologisms and/the like that are useful for drawing conclusions about the patient's then-current behavior, according to predetermined logic. By way of example, the voice data may be processed to identify and/or analyze image features, etc. useful for drawing conclusions about the patient's truthfulness, distress level, and risk of a suicide attempt or another ER visit in the near term, if the patient were to be discharged. The system may also examine important features such as whether the patient is developing trust in the clinician and whether the clinicians are aligning their voices in a way to enhance a therapeutic relationship with the patient to more likely lead to trust and clinical success. By way of further example, voice data can be used, as others have shown, to examine various clinical status metrics. Unlike prior art approaches, the present invention leverages such voice data metrics/conclusions to inform interactions in a live patient/clinician clinical session, e.g., to guide the clinician as to whether a clinician should slow down or whether there is elevated risk of a future suicide attempts or other clinical issues. By way of alternative example, such voice data can also be used to track patient or clinician fatigue, anxiety tied to certain topics, or distraction among other areas. The system thereby enhances and augments the clinician and patient interaction session and increases the clinician's human perceptions of affect and mood in the interaction.

Notably, facial expression/camera data and voice data and may be similarly gathered by the system and similarly may be used by the system, and be processed to cause the system to provide output to at least one of the clinician and the patient to influence/guide the clinician/patient interaction session.

The exemplary embodiment of the PACG System 200 shown in FIG. 2 also includes medical record data 224c stored in the data store 224 of the PACG 200. The medical record data 224c may be health record and/or medical record data for the patient, gathered from the EMR System 120 by communication of the PACG 200 with the EMR System 120 via the communications network 50. Accordingly, prior health/medical record data may be gathered during the patient's operation of the computing device 100a, 100b, 100c, 100d to answer or respond to a prompt 154 displayed to the patient in a graphical user interface window 150 on a display device 114 of the computing device, e.g., 100d, as will be appreciated from FIG. 3. In this embodiment, the SSE 230 further includes a Medical Record Analysis Module 250. The Medical Record Analysis Module 260 is responsible for processing the medical record data 224c to identify information that is useful for understanding the patient's health/medical history. For example, information such as physiological and biological measurements, such as a Chem 7 finding, a CBC findings, a heart rate, a blood pressure, a blood oximetry, a blood glucose, a body temperature, a body fat, a body weight, a sleep duration, a sleep quality, and an electroencephalogram, information relating to use of medications and substances with behavioral or cognitive effects selected from the group consisting of: cocaine, opiates, amphetamines, stimulants and cannabis, information relating to food and diet information, information relating to a dosage, a frequency, and a duration of a medication, information relating to prior hospitalizations, information relating to prior diagnoses, and the like may be useful. By way of example, this information may be identified by processing the data in any suitable manner. By way of example, natural language searching for predefined terms of interest and/or searching for ICD-9 codes of interest may be used. By way of example, the medical record data may be processed to identify and/or analyze medical record data 224c to identify information that is useful for guiding questions/discussion or otherwise vetting the patient's responses to prompts during the clinical patient health assessment, etc., according to predetermined logic.

The exemplary embodiment of the PACG System 200 shown in FIG. 2 also includes an SSE 230 including a Passive Input Interpretation Module (PIIM) 270. The PIIM 270 is responsible for interpreting the results of the facial analysis performed on the camera data 224a by the Facial Analysis Module 140, the results of the voice analysis performed on the voice data 224b by the Voice Analysis Module 250, and the results of the medical records analysis performed on the medical record data 224c by the Medical Record Analysis Module 260. For example, the PIIM 270 may draw inferences or conclusions based on these analyses. For example, the PIIM 270 may draw a conclusion that the patient is being truthful or untruthful, or that the patient is relaxed or distressed, or that there is evasiveness in relation to the patient's intent to harm himself/herself if discharged, or the patient's compliance with their medication regimen. By way of further example, a conclusion that the patient is distressed may cause the system to provide an alert to the clinician that the patient may not understand what the clinician is saying, so that the clinician can take appropriate action. Further, the PIIM 270 may draw conclusions about the patient's health and/or may draw conclusions that may be used to guide a clinician or provide feedback to inform the system as to how to select next prompts/questions to be posed to the patient.

With respect to the Facial Analysis Module 240, the Voice Analysis Module 250, the Medical Records Analysis Module 260 and the Passive Input Interpretation Module 270, it will be recognized that various signal analysis, data analysis, pattern matching, machine learning and artificial intelligence approaches may be employed to identify any suitable features, as desired, and any suitable methodologies and/or algorithms may be used, as desired, as will be appreciated by those skilled in the art.

The exemplary embodiment of the PACG System 200 shown in FIG. 2 also includes Patient Prompt Data 224d stored in the data store 224. The Patient Prompt Data 224d may include questions, sets of questions, and prompts in formats other than questions, that may be used by the system to gather information from the patient during a patient clinical assessment session. Preferably, the Patient Prompt Data 224d includes questions/prompts predefined and prepared to be in accordance with hospital procedures, best practices and/or governing and/or thought-leading bodies, such as the Joint Commission for Hospitals.

The exemplary embodiment of the PACG System 200 shown in FIG. 2 also includes an SSE 230 including a Patient Chat Module (PCM) 280. The PCM 280 is responsible for selecting suitable prompts from the Patient Prompt Data 224d, and for causing display of selected prompts to the patient via the computing device being used by the patient during the clinical patient assessment session. As discussed above, the prompts may be selected at least in part due to predefined logic for presenting prompts sequentially. Further, the prompts may be selected at least in part due to predefined logic for presenting prompts as a function of responses obtained from the patient to one or more previously-displayed prompts. Further, the prompts may be selected at least in part due to the results of interpretations of camera data 224a, voice data 224b and/or medical record data 224c performed by the PIIM 270 and/or the FAM 240, VAM 250 and/or MRAM 260. FIG. 3 shows an exemplary computing device 100d displaying on its display device 114 a graphical user interface window 150 including a patient prompt 152 (“Have you ever had suicidal thoughts?”), and responsive YES/NO patient prompts 154, 156 selectable by the user to provide a response to the patient prompt 152.

The exemplary embodiment of the PACG System 200 shown in FIG. 2 also includes an SSE 230 including a Clinician Chat Module (CCM) 290. The CCM 290 is responsible for selecting suitable prompts from the Clinician Prompt Data 224e, and for causing display of selected prompts to the clinician via the computing device being used by the clinician during the clinical patient assessment session. The prompts may be selected at least in part due to predefined logic for presenting prompts sequentially. Further, the clinician prompts may be selected at least in part due to predefined logic for presenting prompts as a function of responses obtained from the patient to one or more previously-displayed patient prompts. Further, the clinician prompts may be selected at least in part due to the results of interpretations of camera data 224a, voice data 224b and/or medical record data 224c performed by the PIIM 270 and/or the FAM 240, VAM 250 and/or MRAM 260.

FIG. 3 illustrates an exemplary graphical user interface displayable by the PACG System 200 for providing a shared patient/clinician session via a single display screen 114 of a single computing device 100d in accordance with an exemplary embodiment of the present invention. As shown in FIG. 3, an exemplary computing device 100d displays on its display device 114 a graphical user interface window 110 including a clinician prompt 112 (“Discussion topics: Childhood, Adulthood”, etc.). The clinician prompt 112 may be viewed by the clinician during the patient clinical assessment session to provide the clinician with additional information that the clinician may use during the patient clinical assessment session to interact with the patient to perform a more accurate patient clinical assessment.

In this embodiment, both the patient and the clinician are viewing a single computing device 100d concurrently. Accordingly, in this exemplary embodiment, the clinician prompts may be displayed in a subtle and/or coded fashion, such that the meaning of the prompts are more readily apparent to the clinician than the patient and/or presented in a way that may be less disturbing to the patient, since prompts to the clinician will be readily visible to the patient. The clinician can also place specific pieces of information in diagrams. For example, the clinician can select phrases a patient uses and place them in a worksheet or interactive graphic for later reference.

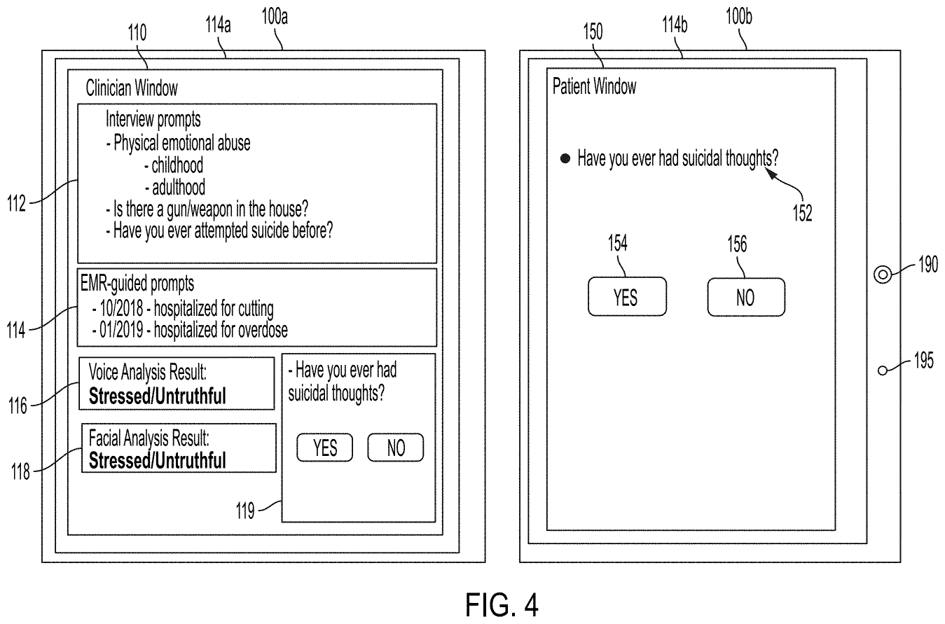

FIG. 4 illustrates an exemplary graphical user interface displayable by the PACG System 200 for providing a shared patient/clinician session via multiple display screens 114a, 114b of multiple computing devices 100a, 100b in accordance with an alternative exemplary embodiment of the present invention. As shown in FIG. 4, the exemplary computing device 100a displays on its display device 114a a graphical user interface window 110 including a clinician prompt 112 (“Interview prompts:—Physical emotional abuse”, etc.). The clinician prompt 112 may be viewed by the clinician during the patient clinical assessment session to provide the clinician with additional information that the clinician may use during the patient clinical assessment session to interact with the patient to perform a more accurate patient clinical assessment.

In this embodiment, the patient and the clinician are using and viewing separate computing devices 100a, 100b concurrently. For example, one of the patient and clinician can see the user interface/display screen of the other if they are in remote locations communicating via video or audio or text. Accordingly, in this exemplary embodiment, the clinician prompts may be displayed to the clinician in an explicit, uncoded fashion, as the prompts to the clinician will not be readily visible to the patient. For instance a prompt may be displayed by the system to suggest possible things to say or activities to suggest that the patient do later, or at that moment. In addition, the system can suggest to the clinician areas to inquire more about.

Accordingly, patient prompts and patient responses provided directly from the patient may be reproduced or “mirrored” and displayed to the clinician via a replica window 119. Additionally, the actively-provided patient responses are supplemented with passively-gathered patient data, and used to guide the questioning of the patient via the computing device and/or to guide the clinician in interacting with the patient, to perform better patient clinical assessments. For example, the clinician window 110 may include a clinician prompt panel 112 based at least in part on information retrieved from the clinician prompt data 224e. Accordingly, when the patient is being prompted with a certain prompt via the patient's computing device 100b, and that certain patient prompt and any response is concurrently being displayed in the replica window 119 on the clinician computing device 100a, the Clinician Chat Module 290 of the SSE 230 may concurrently cause display of related clinician prompts in the clinician prompt window 112. These clinician prompts may be based at least in part on clinical prompt data 224e and/or patient responses actively provided to the PACG System 200 in response to the patient prompts, and may be used to guide the clinician in interacting with the patient during the clinical patient assessment session, to perform better patient clinical assessments.

Additionally, when the patient is being prompted with a certain prompt via the patient's computing device 100b, and that certain patient prompt and any response is concurrently being displayed in the replica window 119 on the clinician computing device 100a, the Clinician Chat Module 290 of the SSE 230 may concurrently cause display of related EMR-guided prompts in the EMR prompt window 114. These EMR prompts may be based on analysis and/or interpretations of medical record data for the patient performed by the Medical Record Analysis Module 260 and/or PIIM 270, and may be used to guide the clinician in interacting with the patient during the clinical patient assessment session, to perform better patient clinical assessments. Analysis and/or interpretations of the medical record data performed by the Medical Record Analysis Module 260 and/or PIIM 270 may also be used to guide and cause display of clinician prompts in the clinician prompt window 112.

Additionally, when the patient is being prompted with a certain prompt via the patient's computing device 100b, and that certain patient prompt and any response is concurrently being displayed in the replica window 119 on the clinician computing device 100a, the Clinician Chat Module 290 of the SSE 230 may concurrently cause display of a Voice Analysis Result in the Voice Analysis prompt window 116. The Voice Analysis prompts may be based on analysis and/or interpretations of voice data for the patient performed by the Voice Analysis Module 250 and/or PIIM 270, and may be used to guide the clinician in interacting with the patient during the clinical patient assessment session, to perform better patient clinical assessments. Analysis and/or interpretations of the voice data performed by the Voice Analysis Module 250 and/or PIIM 270 may also be used to guide and cause display of clinician prompts in the clinician prompt window 112.

Additionally, when the patient is being prompted with a certain prompt via the patient's computing device 100b, and that certain patient prompt and any response is concurrently being displayed in the replica window 119 on the clinician computing device 100a, the Clinician Chat Module 290 of the SSE 230 may concurrently cause display of a Facial Analysis Result in the Facial Analysis prompt window 116. The Facial Analysis prompts may be based on analysis and/or interpretations of camera data for the patient performed by the Facial Analysis Module 240 and/or PIIM 270, and may be used to guide the clinician in interacting with the patient during the clinical patient assessment session, to perform better patient clinical assessments. Analysis and/or interpretations of the camera data performed by the Facial Analysis Module 240 and/or PIIM 270 may also be used to guide and cause display of clinician prompts in the clinician prompt window 112.

All patient and clinician prompts and all responses may be logged by the Patient Chat Module 280 and/or the Clinician Chat Module 290. This information may be stored as raw Patient Assessment Data 224f in the data store 224 of the PACG System 200. Additionally, the SSE 240 includes a Reporting Module 300. The Reporting Module is responsible for gathering data from the patient and clinician prompts and responses and/or for gathering other data from the patient and/or clinician, via their display devices, so create a report as documentation of the patient clinical assessment. This may be performed according to any desired report format, and is preferably performed according to a predefined format that is compatible with best practices, industry guidelines, or the like. These final reports, and any associated safety plans, etc., may be stored as final patient assessment documentation in the Patient Assessment Data 224f of the data store 224 of the PACG System 200.

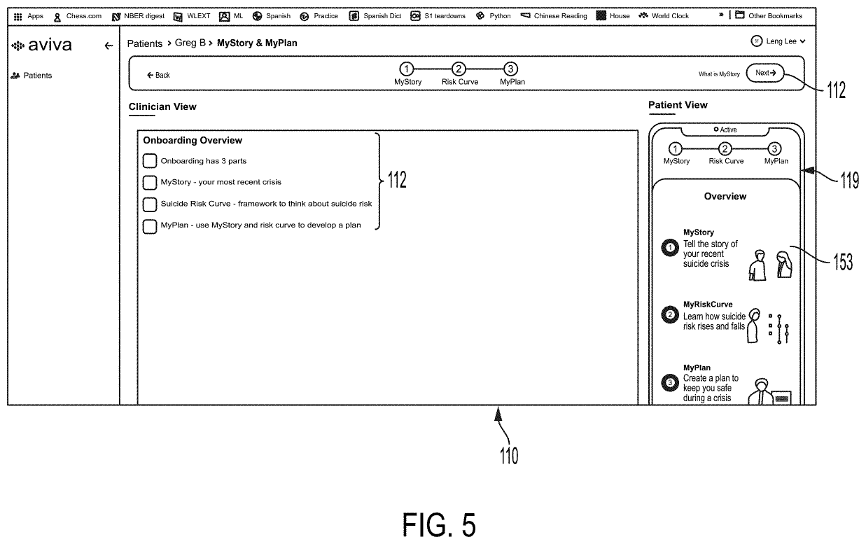

FIGS. 5-20 illustrate another exemplary graphical user interface displayable by the PACG System 200 for providing a shared patient/clinician session via multiple display screens 114a, 114b of multiple computing devices 100a, 100b in accordance with an alternative exemplary embodiment of the present invention. In FIGS. 5-20, only the clinician computing device 100a is shown, but the patient computing device 100b displays a graphical user interface window 150 matching or corresponding closely to the Patient View graphical user interface replica window 119 shown as part of the Clinician View user interface window 110 in FIGS. 5-20.

As shown in FIG. 5, the exemplary computing device 100a displays on its display device 114a a graphical user interface window 110 including a clinician prompt 112. The clinician prompt 112 may be viewed by the clinician during the patient clinical assessment session to provide the clinician with additional information that the clinician may use during the patient clinical assessment session to interact with the patient to perform a more accurate patient clinical assessment, to provide guidance/counsel to the patient, to interactively gather information from the patient and collaboratively document the patient's crisis, and to collaboratively prepare a crisis action plan specific to the patient, so that the patient can refer to and use the crisis action plan (e.g., via the patient computing device) between patient sessions with the clinician.

In this embodiment, as in the embodiment described with respect to FIG. 4, the patient and the clinician are using and viewing separate computing devices 100a, 100b concurrently. The patient and clinician may be located remotely from one another, and in a telemedicine-type consultative session. Clinician input provided via the Clinician View window 110 may be reproduced or “mirrored” and displayed to the patient via a Patent View user interface window 150 displayed on the patient's computing device 100b. In this embodiment, the information content displayed on the patient's computing device is also reproduced or “mirrored” and displayed to the clinician via the replica window 119 portion of the Clinician View window 110. Accordingly, the clinician can control what is displayed at the patient's computing device 100b, in real time, by providing input to the clinicians' device 100a, and while also being provided with a display of a replica window 119 at the clinician's device 100a that displays matching or closely corresponding content to what the patient is shown by a display by the patient computing device 100b. Similarly, patient prompts and/or patient responses provided directly from the patient via the patient's computing device 100b may be reproduced or “mirrored” and displayed to the clinician via the replica window 119 at the clinician's computing device 100a.

In this embodiment, the patient and computing devices are provided via an internet/web-based web socket-type data communication session between the clinician device 100a and the patient device 100b. As known in the art, a typical HTTP request/response data communication exchange is essentially a one-time request for data from a client device to a server device, and a corresponding one-time response. as further known in the art, a web socket is somewhat like an HTTP request and response, but it does not involve a one-time data request and a one-time data response. Rather, the web socket effectively keeps open the data communication channel between the client device and the server device. More particularly, the web socket is essentially a continuous bidirectional internet connection between the client and server that allows for transmission/pushing of data to the other computer without that data first being requested in a typical http request. Accordingly, the web socket is usable for live-syncing of data between multiple devices, because each client/server computer can choose when to update the other, rather than waiting for the other to request it. Accordingly, actively-provided patient input is provided to and displayed at the clinician device 100a, and actively-provided clinician input is provided to and displayed at the patient device 100b. Accordingly, changes input (and/or approved for publication) by the clinician, are then displayed on the patient's device almost immediately, in “real time.” This facilitates collaboration of the clinician and patient in accurately documenting crisis events, in developing a crisis plan, and in sharing information.

Additionally, the actively-provided patient responses may be supplemented with passively-gathered patient data, and be used to guide the questioning of the patient via the computing device and/or to guide the clinician in interacting with the patient, to perform better patient clinical assessments, in a manner similar to that described above. All patient and clinician prompts and all responses may be logged by the Patient Chat Module 280 and/or the Clinician Chat Module 290, etc., in a manner similar to that described above.

Referring now to FIGS. 5-20, exemplary Clinician View windows 110 are shown, including a Patient View replica window 119 that shows information content that is displayed remotely at a patient window 150 at a patient's computing device 100b. The Clinician View window 110, displayed to a clinician on the clinician computing device 100a, allows the clinician to view information content and prompts that are not visible to the patient at the patient computing device 100b, while also communicating with the patient, e.g., via a telephone call, to collaboratively gather/record information from the patient (e.g., MyStory) and counsel the patient while also collaboratively developing additional information content such as a crisis action plan for the patient (e.g., MyPlan). Accordingly, the system provides a collaborative patient assessment and planning tool that can be useful to clinicians to simulate or otherwise be a substitute for what might occur in an in-person, face-to-face, clinician/patient counseling session. Further, the system provides that the action plan so developed remains available to and accessible by the patient, e.g. via the patient's computing device 100b (e.g., via a suitable software “app”) so that the patient may use the crisis action plan at a time when the patient does not have direct access to the clinician, e.g., between clinician consultation sessions.

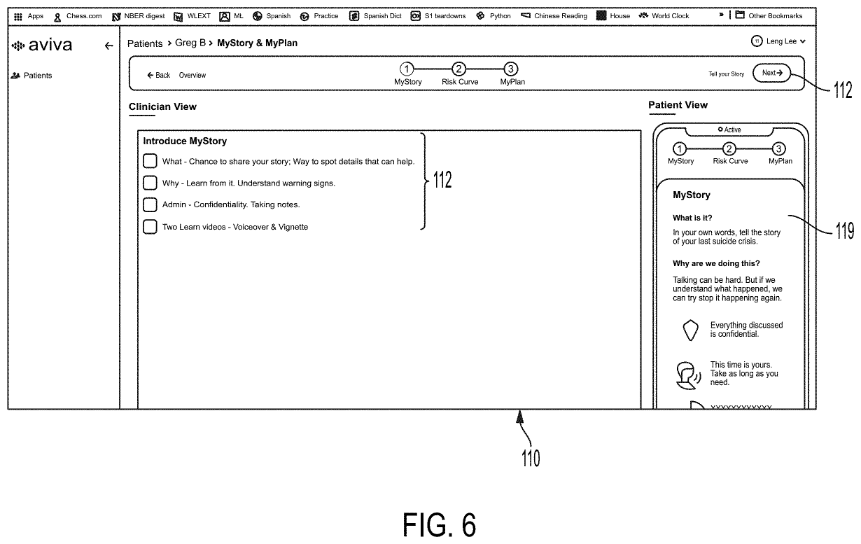

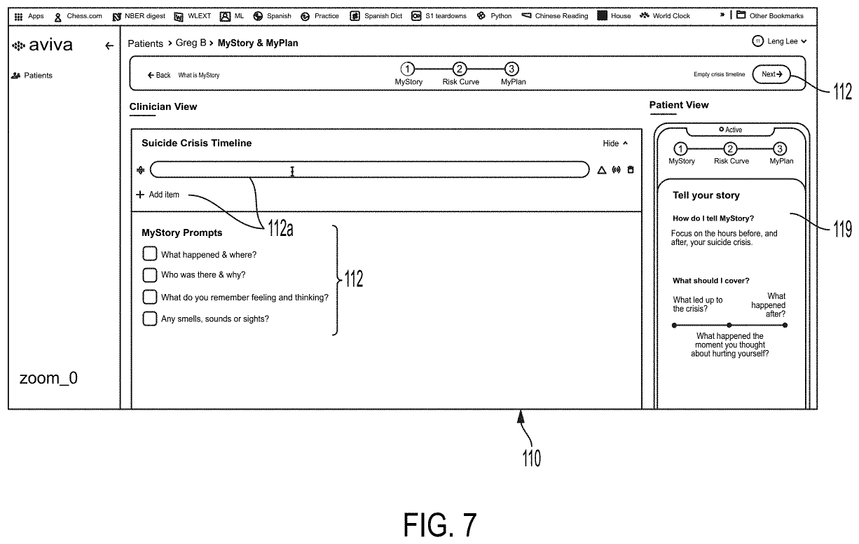

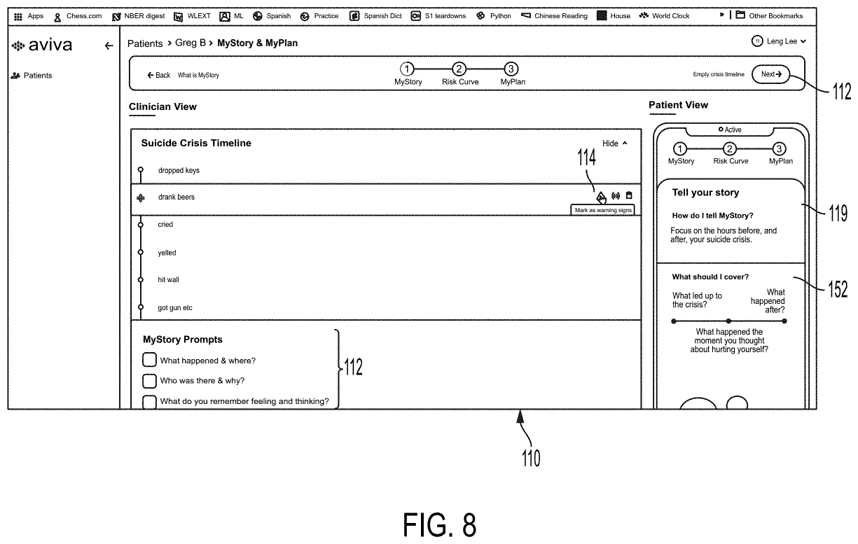

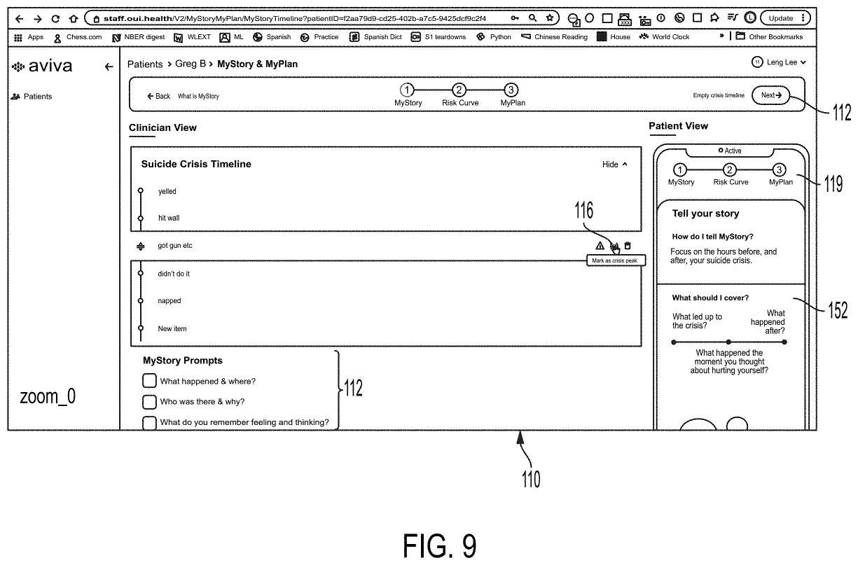

More particularly, the clinician window 110 of FIGS. 5-20 display information content/prompts 112 that guide the clinician in speaking with/consulting with the patient, while the clinician can see the information content/patient prompts 152 displayed at the patient computing device 110b, since the information content/patient prompts 152 are reproduced in the replica window 119 of the clinician window 110 at the clinician device 100a. The clinician windows 110 of FIGS. 5 and 6 display information allowing the clinician to guide the patient through familiarization with the MyStory portion of the information content 152, as displayed in the replica window 119, and to the patient via the patient computing device 100a, these displays being synchronized and mirrored/replicated in real time (e.g., when a change is made on the clinician end, in is promptly reflected in the replica window 119 and at the patient computing device 110b. Accordingly, the clinician and patient can collaboratively review parts of a patient-facing “app” (and associated information content) that provides information that may be referenced by, and be helpful to, the patient outside of a clinician/patient counseling session. As part of the MyStory information content workflow, the system then provides prompts 112a, via the window 110, to gather information relating to actions/events in the patient's crisis to be addressed, e.g., in a recent suicide crisis event. In this example, the clinician can select the Add Item graphical user interface element, and then provided typed or other descriptions of events that occurred during the suicide crisis, e.g., according to information gathered from the patient verbally, e.g., over the telephone, as shown in FIG. 7. In this example, according to information gathered from the patient, the clinician has recorded that the recent patient crisis involved patient events including “dropped keys,” “drank beers,” “cried,” “yelled,” “hit the wall,” “got gun,” “didn't do it,” and “napped,” as shown in FIGS. 8 and 9. For example, these may be clinician-captured descriptions of events provided by the patient in recounting a recent patient crisis.

Further, the clinician and patient can collaboratively (e.g. via a telephone discussion) discuss which of those events are considered to be a characteristic warning sign for the patient's crisis, and the clinician may select a warning sign-marker graphical user element 114 associated with a corresponding patient event to flag such an event as a warning sign in the particular patient's crisis. Here, the “drank beers” patient event has been marked as a warning sign by selecting the warning sign-marker graphical user element 114 associated with the “drank beers” patient event, as shown in FIG. 8. During this time, patient prompts 152 may be displayed as information content on the patient computing device 100b, so the patient can review and verify the documentation in “real time.” As described above, that information content (as displayed to the patient) is displayed reproduced in the replica window 119 in the clinician window 110 on the clinician computing device 100a, as shown in FIG. 8.

As the list of patient events is created by the clinician via input via the clinician computing device 100a, and displayed in the clinician window 110, corresponding information content, in this case a suicide crisis timeline, is displayed as information content 152 on the patient's computing device 100b, and also in the replica window 119 showing in the clinician window 110 what the patient is viewing at that time on the patient computing device 100b.

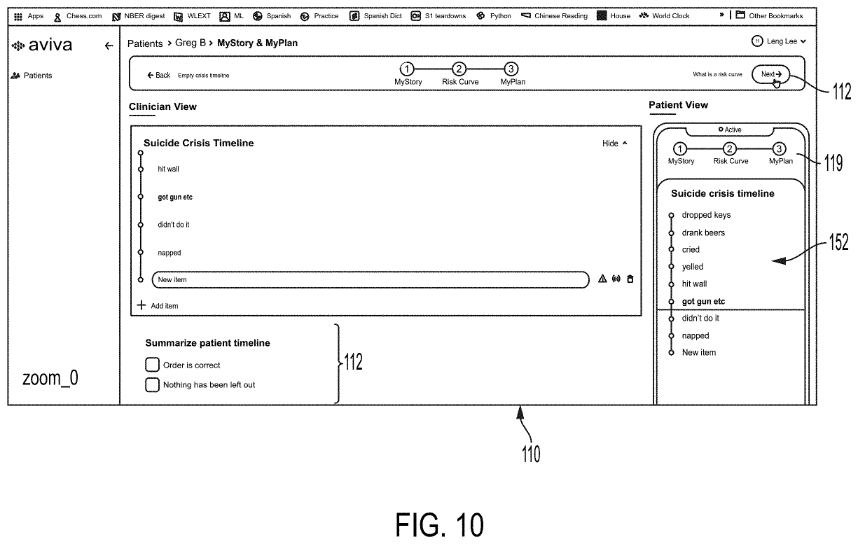

Somewhat similarly, the clinician and patient can collaboratively (e.g. via a telephone discussion) discuss which of those events is considered to be associated with a peak of the crisis, and the clinician may select a peak-marker graphical user element 116 associated with a patient event to flag such an event as a peak in the particular patient's crisis. Here, the “got gun” patient event has been marked as a crisis peak by selecting the peak-marker graphical user element 116 associated with the “got gun” patient event, as shown in FIG. 9. During this time, patient prompts 152 may be displayed as information content on the patient computing device 100b, so the patient can review and verify the documentation in “real time.” As described above, that information content (as displayed to the patient) is displayed reproduced in the replica window 119 in the clinician window 110 on the clinician computing device 100a, as shown in FIG. 10.

Responsive to marking of a particular patient event as the crisis timeline peak, the graphical user interface maps those events to a risk curve showing the patient event marked as a crisis peak at the peak of the risk curve. As shown in FIG. 10, the mapping may be depicted using a color scheme that provides for color-coding of the events to map the events to the risk curve. By way of example, the color scheme may provide that the peak is shown by color of the greatest intensity, darkness, boldness or shading, with correspondingly increasing intensity/darkness/boldness/shading leading up to the peak, and decreasing intensity/darkness/boldness/shading trailing away from the peak. In FIG. 10, this color-coding of events to show a mapping of the risk curve is shown in the clinician window 110, the replica window 119, and in the patient window 150 of the patient computing device, as will be appreciated from FIG. 10.

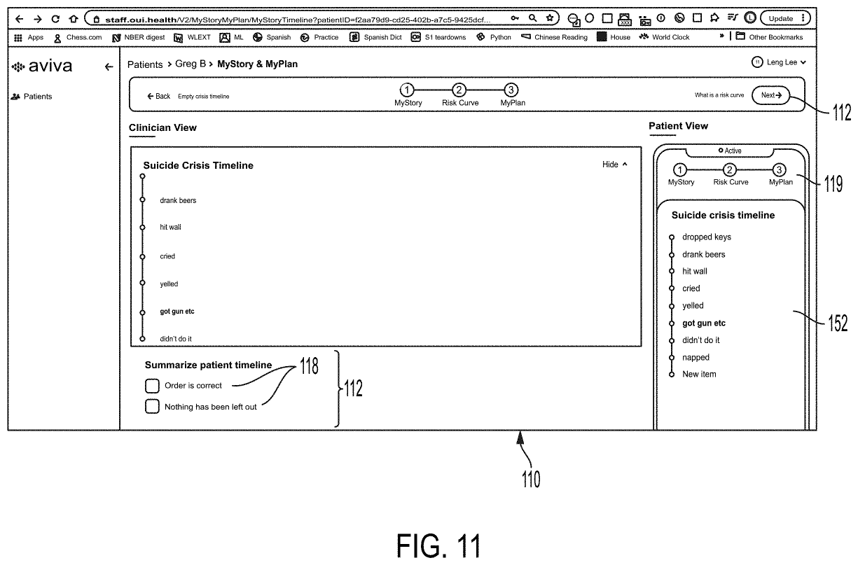

The Clinician View window 110 also provides the clinician with drag-and-drop functionality so that the clinician can easily reorder patient events listed in the suicide crisis timeline. This may be necessary, for example, if the patient, after reviewing the timeline as documented and displayed on the patient computing device 100b (and also shown in the replica window 119 at the clinician computing device 100a) determines that the order of patient events is not accurately depicted/recorded. As will be appreciated from FIG. 11 in FIG. 10, the drag-and-drop functionality of the clinician window 110 has been used to reorder the “got gun” patient event from after “hit wall” to after “yelled.” The risk curve depiction is automatically updated accordingly, as is the display of information content at the patient computing device 100b and in the replica window 119. This facilitates collaboration and documentation of the suicide crisis timeline with the input of both the clinician and the patient, even when the clinician and patient are remotely located and using two different computing devices.

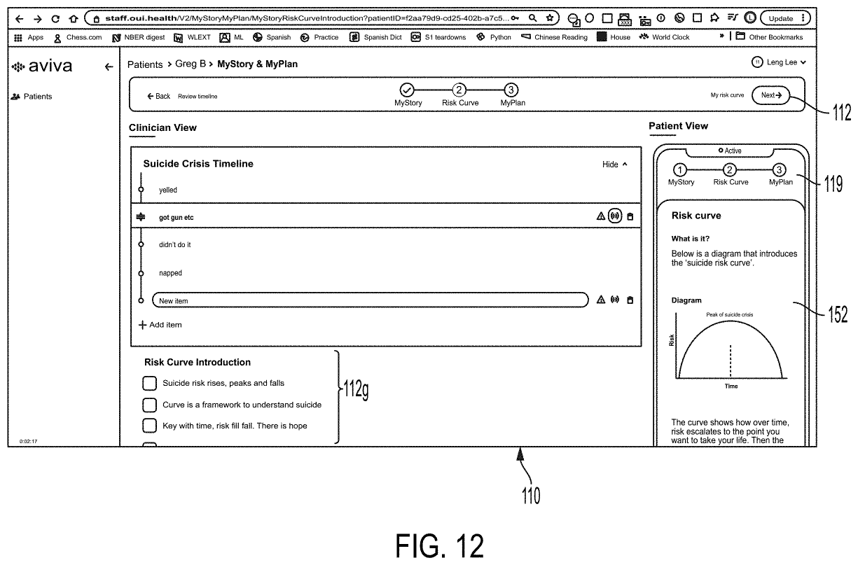

After confirming that the order is correct and that nothing has been left out (e.g. using confirmation graphical user interface elements 118 displayed in the clinician view window 110) the crisis timeline and associated patient events may be mapped to a graphical depiction of the risk curve. Information content providing information about a risk curve generally may be displayed at the patient computing device 100b (and also be reproduced in the replica window 119 of the clinician view window 110 on the clinician computing device 100a) while the clinician is displayed prompts 112g, via the clinician window 110, guiding the clinician through discussion of the risk curve with the patient, as shown in FIG. 12. This allows the clinician and patient to collaboratively review information content accessible via the “app” and/or viewable via the patient device.

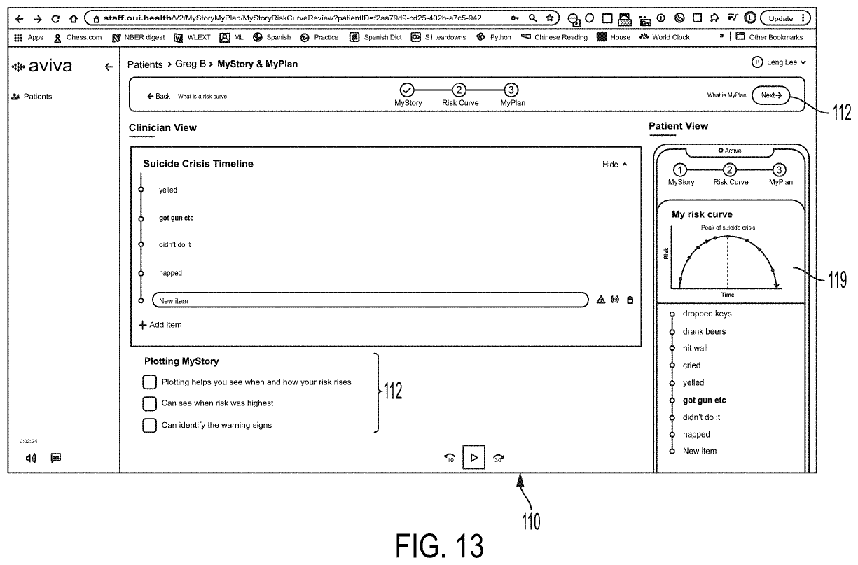

After helping the patient to understand risk curves generally, the system causes display of the particular suicide crisis timeline and associated patient events, gathered/recorded as part of MyStory, mapped to a graphical depiction and/or color-coded depiction of a risk curve, as shown in FIG. 13. Information content displaying the patient-specific risk curve may be displayed at the patient computing device 100b (and also be reproduced in the replica window 119 of the clinician view window 110 on the clinician computing device 100a), as shown in FIG. 13.

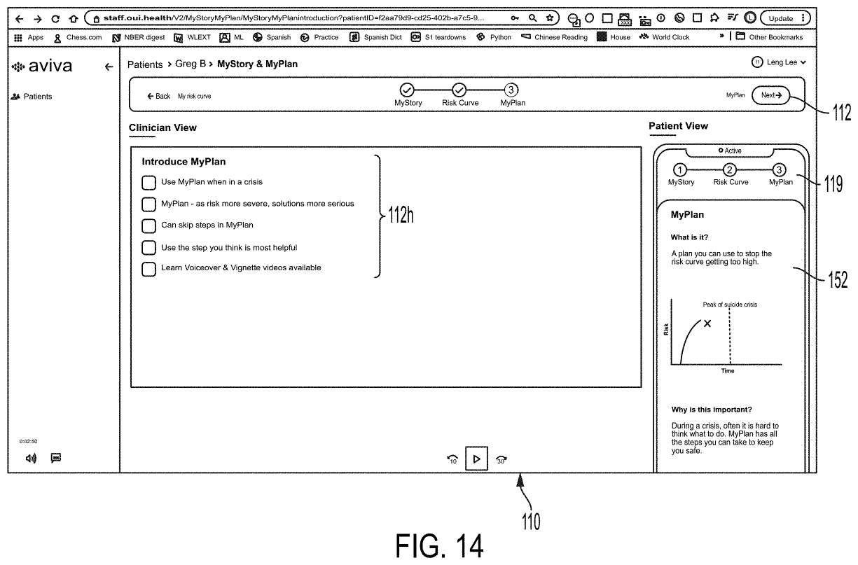

Next, the clinician view window 110 allows the clinician to view information content and prompts that are not visible to the patient at the patient computing device 100b, while also communicating with the patient, e.g., via a telephone call, to collaboratively gather/record information from the patient in developing a crisis action plan for the patient (e.g., MyPlan) as shown in FIG. 14. Information content 152 relating to a crisis action plan generally may be displayed via the patient computing device 100b (and may be reproduced via the replica window 119 of the clinician window 110), as prompts 112h are displayed via the clinician window 110 to guide the clinician through discussion and development of a crisis action plan with the patient, as shown in FIG. 14.

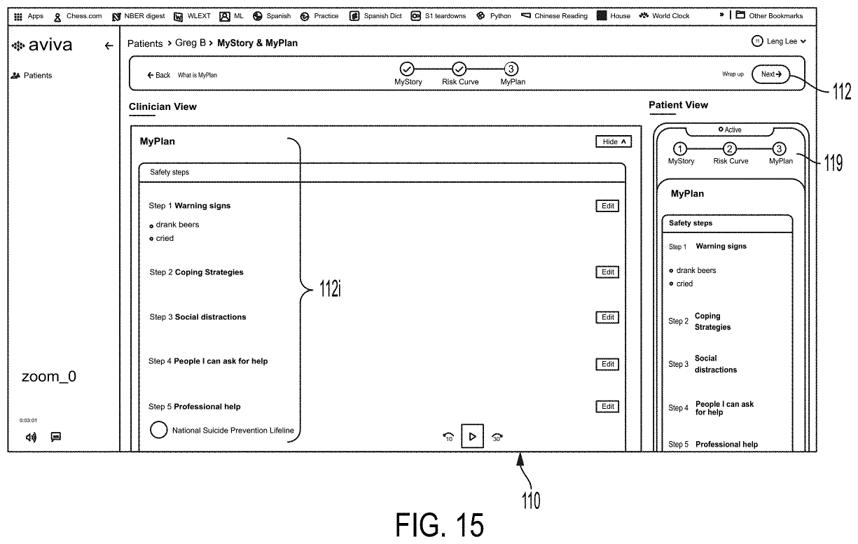

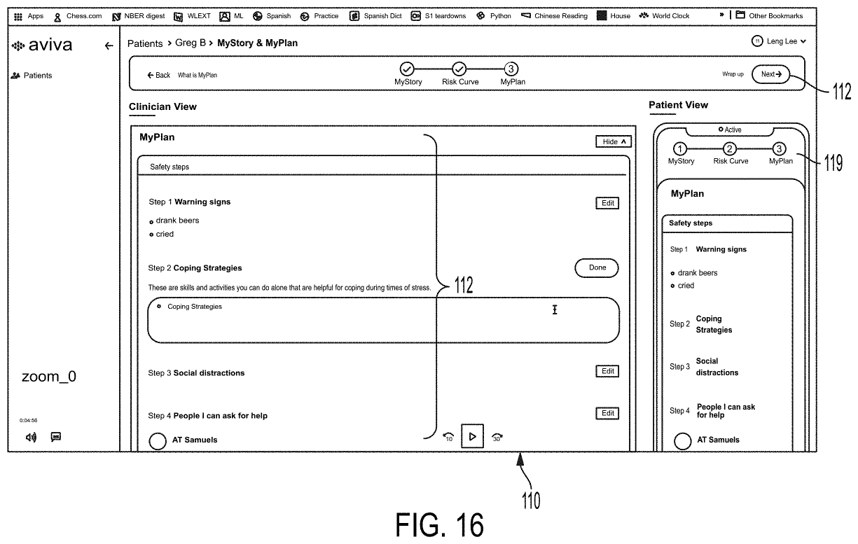

After helping the patient to understand crisis action plans generally, the system causes display of information relating to development of a crisis action plan (e.g., MyPlan), as shown in FIG. 15. More particularly, as part of the MyPlan information content workflow, the system then provides prompts 112i, via the window 110, to gather information relating to actions to be taken and/or other information usable in a crisis action plan for the patient. First, the clinician window 110 may display information content retrieved from information gathered as part of the MyStory workflow. In this example, the patient-specific warning and crisis peak events are pre-populated and displayed in the Warning Signs section of the MyPlan information content displayed via the clinician window 110, as well as via the patient window 150, and reproduced in the replica window 119. The graphical user interface further allows the addition of text and other information (e.g., by selected the Edit graphical user interface element), and then typing in information that will become part of the patient-specific crisis action plan. In this example, “Play Golf” has been entered by the clinician into a text entry field for Coping Strategies, and is displayed as a recordation of an appropriate coping strategy for this particularly patient, as may be discovered by discussion between the clinician and patient, e.g., via the telephone, as will be appreciated from FIGS. 15 and 16.

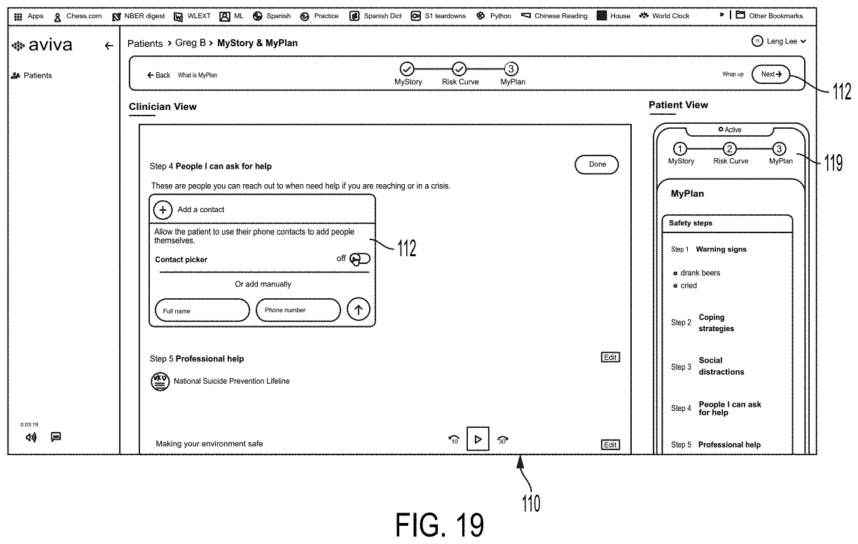

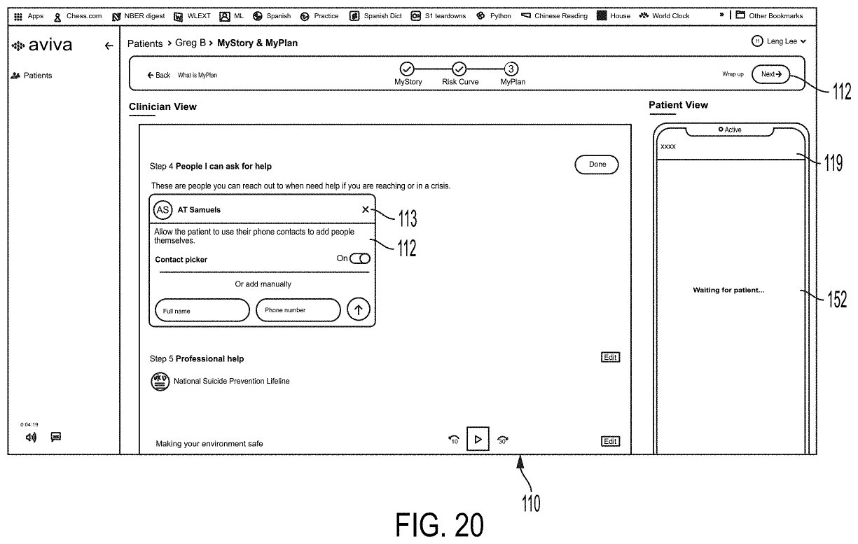

Similarly, information may be added to the patient's crisis action plan using the Edit graphical user interface element provided for Social Distractions, to identify people and places that the patient can use arrange a social event distraction, which may be useful to the patient during a suicide or other crisis. Here, it will be noted that there are prompts 112 and graphical user interface controls usable by the clinician to enable the patient to choose people/contacts from the contact list on the patient computing device. In response to these controls, information context 152 is displayed at the patient's computing device 100b allow the patient to access contact picking functionality, and to add it to the patient's plan. Similar contact-picking functionality is also provided for a People I Can Ask for Help portion of the graphical user interface, as shown in FIG. 19. As shown in FIG. 20, the graphical user interface listing contacts at the patient computing device 100b may not be reproduced in the replica window 119 at the clinician computing device 110, to protect the privacy of the patient. Instead, a blank screen or other generic information content 152 may be displayed in the replica window 119 during the content picking process (in lieu of the contact information viewable at the patient computing device, to protect the patient's privacy), as shown in FIG. 20. After a contact has been selected by the patient, information content identifying the selected contact 113 may be added to a list and may be displayed within the clinician window 110, as shown in FIG. 20.

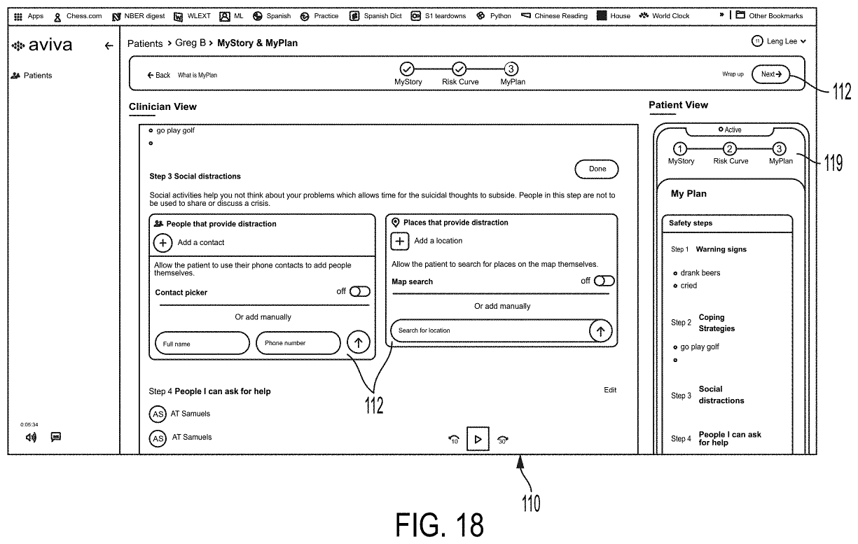

Alternatively, the clinician may type (or otherwise provide) name and telephone number information into text entry boxes of the user interface window to manually add a contact that will become part of the patient's patient-specific crisis action plan, as shown in FIG. 18. Similar functionality may be provided for the People I Can Ask For Help portion of the graphical user interface, as shown in FIGS. 19 and 20.

Additionally, and somewhat similarly, information may be added to the patient's crisis action plan using the Edit graphical user interface element provided for Social Distractions, to identify places that the patient can use arrange a social distraction, which may be useful to the patient during a crisis. Here, it will be noted that there are prompts 112 and graphical user interface controls usable by the clinician to enable the patient to choose a location on a map displayed on the patient computing device. In response to these controls, information content 152 is displayed at the patient's computing device 100b to allow the patient to access location picking functionality, and to add it to the patient's plan, as shown in FIG. 19. After a location has been selected by the patient, information content identifying the selected location may be added to a list and may be displayed within the clinician window 110, as shown in FIG. 18. Additionally, a location may be added manually by a clinician, by typing location information into a text entry box of the clinician user interface window 110, as shown in FIG. 18.

Accordingly, it will be appreciated that the graphical user interface (and system) of the present invention facilitates collaborative interaction of the patient and clinician, even when the patient and clinician are remotely located and using different computing devices, to engage in an interactive and collaborative patient clinical assessment session to perform a more accurate patient clinical assessment, to provide guidance/counsel to the patient, to interactively gather information from the patient and collaboratively document the patient's crisis, and to collaboratively prepare a crisis action plan specific to the patient, so that the patient can refer to and use the crisis action plan (e.g., via the patient computing device) between patient sessions with the clinician.

The various implementations and examples shown above illustrate a method and system for preforming a patient clinical assessment using an electronic device. As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present implementation. Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

In an exemplary embodiment, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a smart phone, a web appliance, a network router, switch or bridge, or any machine capable of executing a set of instructions (sequential or otherwise) that specify actions to be taken by that machine or computing device. Further, while only a single machine is illustrated, the term “machine” shall also be taken to include any collection of machines that individually or jointly execute a set (or multiple sets) of instructions to perform any one or more of the methodologies discussed herein.

The example computer system and client computers include a processor (e.g., a central processing unit (CPU) a graphics processing unit (GPU) or both), a main memory and a static memory, which communicate with each other via a bus. The computer system may further include a video/graphical display unit (e.g., a liquid crystal display (LCD) or a cathode ray tube (CRT)). The computer system and client computing devices also include an alphanumeric input device (e.g., a keyboard or touch-screen), a cursor control device (e.g., a mouse or gestures on a touch-screen), a drive unit, a signal generation device (e.g., a speaker and microphone) and a network interface device.

The system may include a computer-readable medium on which is stored one or more sets of instructions (e.g., software) embodying any one or more of the methodologies or systems described herein. The software may also reside, completely or at least partially, within the main memory and/or within the processor during execution thereof by the computer system, the main memory and the processor also constituting computer-readable media. The software may further be transmitted or received over a network via the network interface device.

The term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the present implementation. The term “computer-readable medium” shall accordingly be taken to include, but not be limited to, solid-state memories, and optical media, and magnetic media.

What is claimed is:

1. A computer-implemented method for patient assessment using a computerized patient assessment and clinician guidance system having at least one processor and a memory operatively coupled to the at least one processor and storing instructions executable by the processor, the method comprising:
creating a shared session comprising a graphical user interface viewable by both a patient and a clinician on at least one computing device, the graphical user interface displaying first information configured for viewing by the patient, and second information configured for viewing by the clinician.

2. The method of claim 1, wherein said at least one computing device comprises a camera, and wherein said patient assessment and clinician guidance system is configured to display to a clinician image data captured by said camera.

3. The method of claim 2, wherein said patient assessment and clinical guidance system comprises a facial analysis module configured to process image data captured by said camera to analyze facial features captured by said image data, to draw conclusions according to predetermined logic based on analysis of facial features, and to display corresponding information to said clinician.

4. The method of claim 1, wherein said at least one computing device comprises a microphone, and wherein said patient assessment and clinician guidance system is configured to play to a clinician voice data captured by said microphone.

5. The method of claim 4, wherein said patient assessment and clinical guidance system comprises a voice analysis module configured to process voice data captured by said microphone to analyze vocal features captured by said voice data, to draw conclusions according to predetermined logic based on analysis of vocal features, and to display corresponding information to said clinician.

6. The method of claim 1, further comprising processing medical record data to identify medical history information useful for vetting a patient's responses to prompts obtained via a clinical patient health assessment according to predetermined logic, drawing a conclusion based on processed medical record data, and displaying corresponding information to said clinician.

7. The method of claim 1, further comprising interpreting data resulting from processing of patient-related data, drawing a conclusion according to predetermined logic based on interpreted data, and displaying corresponding information to said clinician.

8. The method of claim 1, further comprising selecting a prompt to a clinical based on a conclusion drawn by the system and displaying the prompt to said clinician.

9. The method of claim 1, further comprising selecting a question from a set of stored questions based on a conclusion drawn by the system, and displaying the question to said clinician.

10. The method of claim 1, wherein the system displays information to the patient via a first computing device, and wherein the system displays information to the clinician via said first computing device.

11. The method of claim 1, wherein the system displays information to the patient via a first computing device, and wherein the system displays information to the clinician via a second computing device distinct from said first computing device.

12. The method of claim 11, wherein prompts and patient responses displayed on the first computing device are also displayed on the second computing device.

13. The method of claim 11, wherein clinician input provided via the second computing device and displayed on the second computing device is also displayed concurrently via the first computing device in a shared user interface.

14. The method of claim 11, wherein information content displayed to the patient on the first computing device is also displayed concurrently to the clinician in a replica window displayed on the second computing device in a shared user interface.

15. The method of claim 11, wherein the shared user interface is provided on the first and second computing devices via a web socket-type data communication session to allow live-syncing of data between multiple devices.

16. A patient assessment and clinician guidance system comprising:
a processor;
a memory operatively connected to the processor, said memory storing executable instructions that, when executed by the processor, causes the patient assessment and clinician guidance system to perform a method for patient assessment, the method comprising:
creating a shared session comprising a graphical user interface viewable by both a patient and a clinician on at least one computing device, the graphical user interface display first information configured for viewing by the patient, and second information configured for viewing by the clinician.

17. The system of claim 16, wherein said at least one computing device comprises a camera, and wherein said patient assessment and clinician guidance system is configured to display to a clinician image data captured by said camera.

18. The system of claim 17, wherein said patient assessment and clinical guidance system comprises a facial analysis module configured to process image data captured by said camera to analyze facial features captured by said image data, to draw conclusions according to predetermined logic based on analysis of facial features, and to display corresponding information to said clinician.

19. The system of claim 16, wherein said at least one computing device comprises a microphone, and wherein said patient assessment and clinician guidance system is configured to play to a clinician voice data captured by said microphone.

20. The system of claim 19, wherein said patient assessment and clinical guidance system comprises a voice analysis module configured to process voice data captured by said microphone to analyze vocal features captured by said voice data, to draw conclusions according to predetermined logic based on analysis of vocal features, and to display corresponding information to said clinician.

21. The system of claim 16, further comprising processing medical record data to identify medical history information useful for vetting a patient's responses to prompts obtained via a clinical patient health assessment according to predetermined logic, drawing a conclusion based on processed medical record data, and displaying corresponding information to said clinician.

22. The system of claim 16, further comprising interpreting data resulting from processing of patient-related data, drawing a conclusion according to predetermined logic based on interpreted data, and displaying corresponding information to said clinician.

23. The system of claim 16, further comprising selecting a prompt to a clinical based on a conclusion drawn by the system and displaying the prompt to said clinician.

24. The system of claim 16, further comprising selecting a question from a set of stored questions based on a conclusion drawn by the system, and displaying the question to said clinician.

25. The system of claim 16, wherein the system displays information to the patient via a first computing device, and wherein the system displays information to the clinician via said first computing device.

26. The system of claim 16, wherein the system displays information to the patient via a first computing device, and wherein the system displays information to the clinician via a second computing device distinct from said first computing device.

27. The system of claim 26, wherein prompts and patient responses displayed on the first computing device are also displayed on the second computing device.

28. The system of claim 26, wherein clinician input provided via the second computing device and displayed on the second computing device is also displayed concurrently via the first computing device in a shared user interface.

29. The system of claim 26, wherein information content displayed to the patient on the first computing device is also displayed concurrently to the clinician in a replica window displayed on the second computing device in a shared user interface.

30. The system of claim 26, wherein the shared user interface is provided on the first and second computing devices via a web socket-type data communication session to allow live-syncing of data between multiple devices.

31. A computer program product for implementing a method for patient assessment, the computer program product comprising a non-transitory computer-readable medium storing executable instructions that, when executed by a processor, cause a computerized system to perform a method for patient assessment, the method comprising:
creating a shared session comprising a graphical user interface viewable by both a patient and a clinician on at least one computing device, the graphical user interface display first information configured for viewing by the patient, and second information configured for viewing by the clinician.
